(19) United States
(12) Patent Application Publication
(10) Pub. No.: US 2009/0062664 A1
(43) Pub. Date: Mar. 5, 2009

(54) BLOOD PRESSURE MEASUREMENT DEVICE

(75) Inventors: Chin-Hsiung Chang, Tai-Chung (TW); Fu-Chung Yen, Plano, TX (US)

Correspondence Address: Fu Chung Yen, 3952 Sunflower Lane, Plano, TX, 75025, US

(73) Assignee: FEGO Precision Industrial Co., Ltd., Tai-Chung (TW)

(21) Appl. No.: 11/848,025

(22) Filed: Aug. 30, 2007

(51) Int. Cl. A61B 5/021

(52) U.S. Cl. 600/485; 600/300; 600/481; 702/127; 702/139

(57) ABSTRACT

A blood pressure measurement device. The device comprises a measurement component for measuring blood pressure and a display component for displaying the blood pressure data measured. The display component is adapted to display simultaneously at least two sets of blood pressure measurement data measured at different date or time. Alternatively, the device further comprises a measurement state control component for recording and storing at least one measurement state related to the measurement of the blood pressure, and means for providing the at least one measurement state. Alternatively, the device further comprises an interfacing component that comprises a bi-direction multifunctional port, for exchanging blood pressure data measured between the blood pressure measurement device and at least one external device.

### CROSS-REFERENCE TO RELATED APPLICATIONS

This application is related to: U.S. application Ser. No. unknown, filed currently with the present application on Aug. 30, 2007, entitled “SYSTEM FOR INTEGRATING AND MANAGING HEALTH RELATED INFORMATION”, by Chin-Hsiung Chang and Fu-Chung Yen; and U.S. application Ser. No. unknown, filed currently with the present application on Aug. 30, 2007, entitled “SYSTEM FOR PROVIDING ELECTRONIC NOTES IN A HEALTH RELATED MEASUREMENT DEVICE”, by Chin-Hsiung Chang and Fu-Chung Yen.

### TECHNICAL FIELD OF THE INVENTION

The present invention relates generally to electronic devices, and, more particularly, to a blood pressure measurement device with multiple functions.

### BACKGROUND OF THE INVENTION

Diagnosis of hypertension is of great importance in healthcare. Obtaining reliable Blood Pressure (BP) measurements has become one of the major topics in healthcare and people's routine life. Even though more and more people are aware of the importance to check Blood Pressure (BP) regularly and keep BP in a normal range, most people still measure BP only when they visit doctors or use portable BP devices at home to measure BP once every few days or even weeks. In reality, BP level varies with time, and is significantly influenced by many measurement states at the time of measurement. The measurement states may comprise physical or physiological conditions of a measurement subject, such as before or after eating, before or after a fitness exercise, sickness, taking medicine, body temperature, or measurement time, etc; or environmental conditions, such as temperature, humidity, etc; or other factors. Therefore, a single BP measurement without considering measurement states just reflects the BP at a giving moment, which has little value for diagnosing the hypertension. Conventional BP devices may not support functions of displaying, measuring or storing such measurement states.

Long term, regular BP measurements, together with records of measurement states during BP measurements will provide more reliable and objective BP data that is useful for monitoring and understanding blood pressure level variations under different conditions. However, people often encounter at least two problems in blood pressure measurements. One problem is the incorrect or inaccurate BP measurements obtained. If blood pressure is measured at home, the blood pressure readings might not be accurate due to incorrect ways in which a user operates a BP device, or because a user reads or writes down wrong BP values. If blood pressure is measured in a hospital or a clinic, some people may have “White Coat Hypertension” syndrome and demonstrate hypertensive blood pressure only in the hospital or a physician's office.

Another significant issue is the irregular recording of BP measurements. It is not unusual that people forget to measure BP for some period of time, forget to record BP values after measurements, or stop BP measurements for some other reasons, such as difficulty to operate a BP device, or a user may just feel not useful to use a BP device. Therefore it is difficult for a person to keep regularly measuring and recording of BP levels for a period of time. The incorrect, irregular, and short-term blood pressure data may lead inconsistent and incorrect diagnosis of hypertension, which affects possible treatment regimen and life style.

Furthermore, most conventional BP measurement devices do not support communication, such as exchanging data, with other external devices, such as a key board, a display, a storage device, a fitness apparatus, etc. There may be BP measurement devices that may store (write only) BP data in a computer, or an external storage device. Conventional BP measurement devices have limited capabilities to receive various inputs, such as a text or voice message, body or room temperature measurements, etc, and to output in different manners, such as print out via a printer, etc.

Therefore, there is a need of a BP measurement device that eases operation and use of the BP measurement device, and helps people keep regular tracking of BP levels.

There is another need of a BP measurement device that provides input or measurement, display, and storage of measurement states together with BP measurements.

There is a need of a BP measurement device that supports bi-direction communication with various external devices.

### SUMMARY OF THE INVENTION

A blood pressure measurement device is provided. The device comprises a measurement component for measuring blood pressure and a display component for displaying the blood pressure data measured. In one embodiment, the display component is adapted to display simultaneously at least two sets of blood pressure measurement data measured at different date or time. In another embodiment, the device further comprises a measurement state control component for recording and storing at least one measurement state related to the measurement of the blood pressure, and means for providing the at least one measurement state. In an alternative embodiment, the device further comprises an interfacing component that comprises a bi-direction multifunctional port, for exchanging blood pressure data measured between the blood pressure measurement device and at least one external device.

A blood pressure measurement device in any of the above embodiments may further provide real-time guidance for users to use and operate the blood pressure measurement device.

The following description and drawings set forth in detail a number of illustrative embodiments of the invention. These embodiments are indicative of but a few of the various ways in which the present invention may be utilized.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a block diagram depicting an embodiment of a blood pressure measurement device according to the present invention;

FIG. 2 is a block diagram depicting an embodiment of a blood pressure measurement device according to the present invention;

FIG. 3 is a block diagram depicting an embodiment of a blood pressure measurement device according to the present invention;

FIG. 4 is a block diagram depicting an embodiment of a blood pressure measurement device according to the present invention;

FIG. 5A depicts an embodiment of a blood pressure measurement device according to the present invention;

FIG. 5B depicts a housing of the blood pressure measurement device in FIG. 5A according to the present invention;

FIG. 5C depicts an embodiment of buttons for inputting measurement states to the blood pressure measurement device in FIG. 5A according to the present invention;

FIG. 5D depicts an embodiment of a rotary switch of the blood pressure measurement device in FIG. 5A according to the present invention;

FIG. 6 is a block diagram depicting an embodiment of a display screen for the blood pressure measurement device in FIG. 5 according to the present invention;

FIG. 7 is a block diagram depicting an embodiment of component structures of the blood pressure measurement device in FIG. 5 according to the present invention;

FIG. 8 is a flow diagram of the blood pressure measurement device in FIG. 5 according to the present invention;

FIG. 9 is a flow diagram depicting an embodiment for using the blood pressure measurement device in FIG. 5 according to the present invention; and

FIG. 10 is a diagram depicting an embodiment of a system for integrating and managing blood pressure measurement data according to the present invention.

### DETAILED DESCRIPTION

The following discussion is presented to enable a person skilled in the art to make and use the invention. The general principles described herein may be applied to embodiments and applications other than those detailed below without departing from the spirit and scope of the present invention as defined herein. The present invention is not intended to be limited to the embodiments shown, but is to be accorded the widest scope consistent with the principles and features disclosed herein.

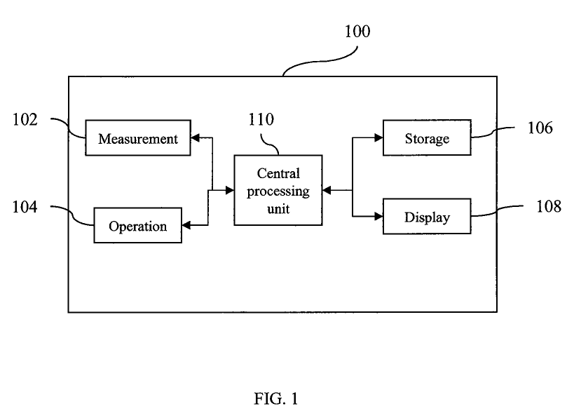

Referring to FIG. 1, a block diagram of a blood pressure measurement device (100) is illustrated according to one embodiment of the present invention. The blood pressure measurement device (100) may display simultaneously multiple sets of blood pressure measurement data at different time.

The blood pressure measurement device (100) may comprise a measurement component (102) for performing measurement of blood pressure; an operation component (104) for operating the blood pressure measurement device (100); a storage component (106) and a display component (108) for storing and displaying, respectively, various data regarding blood pressure measurement; and a Central Processing Unit (CPU) (110) for centrally controlling and monitoring the respective components of the blood pressure measurement device (100).

The display component (108) may be adapted to display simultaneously multiple sets of blood pressure measurement data measured at different date or time. A set of blood pressure measurement data may comprise a systolic pressure value, a diastolic pressure value, and a heart rate value. In one embodiment, the blood pressure measurement device (100) may display two sets of blood pressure measurement data at the same time, e.g., current and previous blood pressure measurement data at the same time. The blood pressure measurement device (100) may have a display screen, which is divided into two sections for simultaneously displaying the current and previous blood pressure measurement, respectively. Before measurement of the current blood pressure, the display component (108) may, under control of CPU (110), obtain previous blood pressure measurement data from the storage component (106) and display in one section of the display screen. After measuring the current blood pressure, the display component (108) may display the current blood pressure measurement data in another section of the display. Thus a user may conveniently and easily compare the current and previous blood pressure measurements.

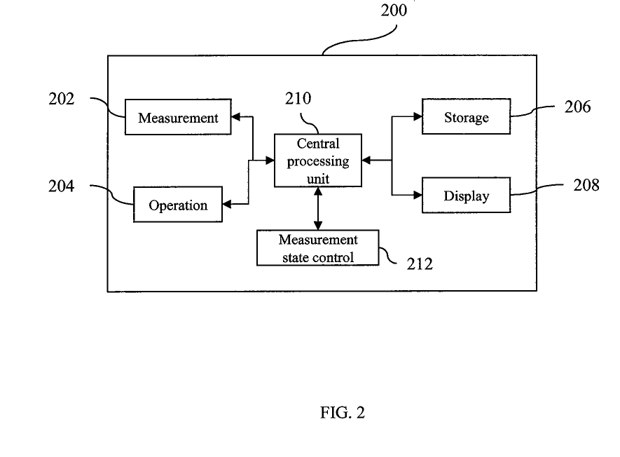

FIG. 2 illustrates another block diagram of a blood pressure measurement device (200) according to one embodiment of the present invention. The blood pressure measurement device (200) may comprise a measurement component (202) for performing measurement of blood pressure; an operation component (204) for operating the blood pressure measurement device (200); a storage component (206) and a display component (208) for storing and displaying, respectively, various data regarding blood pressure measurement; and a CPU (210) for centrally controlling and monitoring the respective components of the blood pressure measurement device (200); and a measurement state control component (212) for recording measurement states corresponding to each blood pressure measurement event and storing the measurement states with the corresponding blood pressure measurement data.

The measurement states may comprise physical or physiological conditions of a user, such as before or after eating, before or after a fitness exercise, before or after taking medicine, emotional conditions, etc., uncomfortable body conditions, such as cough, fever, headache, etc.; environmental conditions, such as temperature, humidity; measurement date or time, or other factors that may affect or may be related to the measurement of the blood pressure.

There may be various ways to input the measurement states with respect to a blood pressure measurement event. In one embodiment, a measurement state may be selected by operating one or more operational keys on the blood pressure measurement device (200). The operational keys may comprise buttons, rotary switches, slide bars, etc. In this instance, this type of measurement states may be represented simply by “yes” or “no”, such as whether a subject has taken medication, or whether a subject has done exercises. Therefore, by operating operational keys, e.g., by pressing a button of the blood pressure measurement device (200), a measurement state for a subject may be selected or deselected. The measurement state may be further classified in severity of degrees, such as extremely/very/kind-of hungry, drink a-lot-of/some/a little coffee, etc. In this case, one or combination of multiple operational keys may be used to record the measurement state.

Measurement states, such as body or room temperature, room humidity, etc, may be measured by a corresponding measurement device, e.g., a body temperature sensor, and recorded with corresponding blood pressure measurement data. The measurement device may be embedded in the blood pressure measurement device (200), or removably connected with the blood pressure measurement device (200) when a particular measurement state needs to be measured.

A measurement state may be measured and recorded automatically, or manually by operating an operational key on the blood pressure measurement device (200), or according to configuration of the blood pressure measurement device (200). For example, a user may removably connect a room temperature sensor with the blood pressure measurement device (200) via an AUX port on the blood pressure measurement device (200). The room temperature will be measured automatically when the blood pressure measurement device (100) detects that a room temperature sensor is connected, and then stored in the storage component (206).

In addition, measurement states may also be collected and recorded by ways of inputting texts, audios, etc from the operational keys of the blood pressure measurement device (200), or from other external devices which may be connected with the blood pressure measurement device (200) when needed. For example, a user just got a flu shot and does not feel well, and the blood pressure measurement device (200) does not provide such state selection. The user may connect a microphone with the blood pressure measurement device (200) via an AUX port, and record the voice description of his/her current physical states in the blood pressure measurement device (200). Alternatively, the user may also connect a computer with the blood pressure measurement device (200), and type in the measurement state. Other external devices that may be used to input measurement states into the blood pressure measurement device (200) include, but not limited to, a keyboard or key pad, etc. Thus more measurement state information may be obtained concerning the blood pressure measurement. By use of the operational keys and other input ways, meaningful measurement states are easily obtained together with the blood pressure measurements, reflecting real conditions of a subject using the blood pressure measurement device (200).

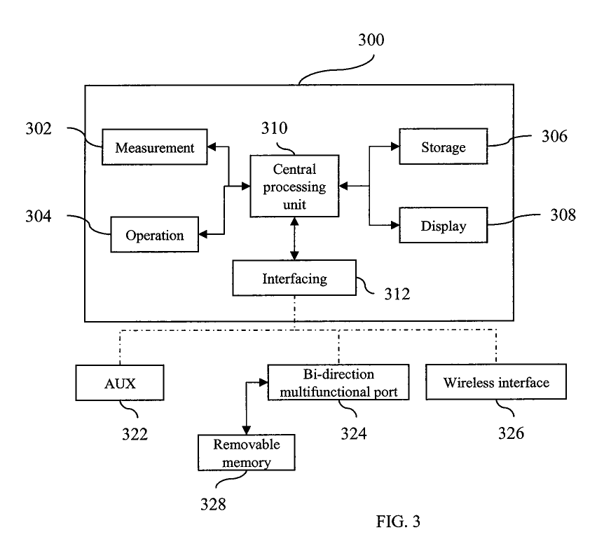

Referring to FIG. 3, a block diagram of a blood pressure measurement device (300) according to one embodiment of the present invention. Similar to the blood pressure measurement device in the embodiment of FIG. 2, the blood pressure measurement device (300) may comprise a measurement component (302), an operation component (304), a storage component (306), a display component (308) and a CPU (310). However, the blood pressure measurement device (300) may not have a measurement state component as in FIG. 2.

The blood pressure measurement device (300) may further comprise an interfacing component (312), through which the blood pressure measurement device (300) may connect and communicate with various external components or devices. These external components or devices may provide measurement of data other than blood pressure, or provide means for entering information related to the blood pressure measurement, such as measurement states, notes, or alarms. In one example, the interfacing component (312) may comprise an AUX port (322), by which, external components, such as a body temperature sensor, a microphone, or a room temperature reader, may be connected with the blood pressure measurement device (300). The blood pressure measurement device (300) may obtain measurement of body temperature of a subject provided by the temperature sensor connected with may provide, and save the body temperature together with blood pressure measurement data.

The interfacing component (312) may comprise a bi-direction multi-functional port (324), by which the blood pressure measurement device (300) may communicate with or exchange data with other external devices. These external devices may include, but not limit to, a removable memory device, for communicating (or exchanging) data with the blood pressure measurement device (300); a printer for printing out data stored in the blood pressure measurement device (300); a keyboard or a microphone, for inputting text or audio information to the blood pressure measurement device (300); a computer or another blood pressure measurement device, for exchanging data with the blood pressure measurement device (300); another display screen for displaying purpose; or other external devices applicable.

The interfacing component (312) may also provide wireless connection with other external devices. For example, the interfacing component (312) may comprise a wireless interface (326), such that the blood pressure measurement device (300) may be connected with a computer wirelessly. A user may use the computer to manipulate data regarding the blood pressure measurement conveniently. The bi-direction multi-functional port (324) may also be a port that provides wireless connection to other external devices.

The interfacing component (312) may greatly extend functions provided by the blood pressure measurement device (300), e.g., the blood pressure measurement device (300) may print out data, display data on a different larger display screen, receives different types of inputs, etc, and improve flexibility for using a blood pressure measurement device. The interfacing component (312) of the blood pressure measurement device (300) may comprise any of the AUX port, bi-direction multi-functional port, and wireless interface, or any combination of the AUX port, bi-direction multi-functional port, and wireless interface if applicable.

A removable memory device, e.g., removable memory device (328) in FIG. 3, when connected with the blood pressure measurement device (300) via the bi-direction multifunctional port (324), may be used to store any data regarding blood pressure measurement of the blood pressure measurement device (300). Each time after measurement, the blood pressure measurement device (300) may automatically or manually store data into the storage component (306), and/or a removable memory device if connected with the blood pressure measurement device (300) via the bi-direction multi-functional port (324). If the embedded memory (306) is full, a user may select to delete the data in the embedded memory (306), overwrite the old data, or store the consequent measurement data in the removable memory device. Thus the storage capacity is extended. Furthermore, a user may flexibly carry with him/her the removable memory device with the data stored.

The removable memory device may be any non-volatile memory, such as flash memory products or any other applicable portable memory devices. The flash memory products may include, but not limited to, flash drives and flash memory cards, etc. One example of the removable memory device is a Universal Serial Bus (USB) flash drive that may get connected via a USB interface. Because of the near-ubiquity of USB support on modern computers and other electronic devices, a USB flash drive may be able to work in many places. In this case the bi-direction multifunctional port (324) may be a USB port. Another example may be a memory stick. When a memory stick is used as the removable memory device, a memory card reader/writer—which may be a build-in component or a peripheral—may be needed for connecting the blood pressure measurement device (300) with the memory stick, reading or writing data.

Since the bi-direction multifunctional port (324), if provided by the blood pressure measurement device (300), may not only be used to connect a removable memory device, but also to connect other removable external devices, such as a printer, or a computer, etc., writing or reading data. It is preferred that the bi-direction multifunctional port (324) is an interface compatible with multiple external devices. One example of such an interface may be a USB port that is supported on many electronic devices and computers.

When using a removable memory device, such as the removable memory device (328) in FIG. 3, as a storage means, software programs may be provided on the blood pressure measurement device (300) such that the removable memory device (328) may not only work as a device for storing and transferring data as already known, but also as a center for integrating data provided by the blood pressure measurement device (300). That is, the blood pressure measurements, and other related data are not simply copied to the removable memory device (328), but saved in a particular format, and stored on the removable memory device (328) in a particular file structure. Thus, the data provided by the blood pressure measurement device (300) may be recorded in more systematic and meaningful forms, which is apparently more useful to users. For example, each time using the blood pressure measurement device (300), the blood pressure measurement device (300) may measure blood pressure, heart rate, etc., obtain or measure other related data, such as measurement states, etc., save the data into a file, and write to the removable memory device (328). Data in the removable memory device (328) may be organized based on different categorization requirements. For instance, the data may be organized based on time, or based on measurement classification, etc.

Table 1 illustrates one embodiment for organizing blood pressure and other related data in the removable memory device (328). In this embodiment, multiple users, i.e., two users, User 1 and User 2, share one removable memory device (328). For example, two household members use one USB flash drive for storing their blood pressure and other related data. In this case, firmware on the blood pressure measurement device (300) may provide mechanism to distinguish one user from the other. Each user may have one independent main folder created on the USB flash drive, each main folder having a different name, such as the main folders [Health_Care_Info User_{—}1] and [Health_Care_Info_User_{—}2] in Table 1. One main data folder may be created for a single user to contain various health related data of the user, and subfolders may be created under the main data folder to contain the blood pressure and other related data according to a specific organizational structure and format for the user. All subfolders and files related to the blood pressure of a user may be placed under a main data folder corresponding to the user. The files in the main data folder or subfolders may be text files, image files, audio files, video files, or may be in any other applicable formats.

Each user may be assigned a unique key or an ID, which may be saved in the removable memory device (328), i.e., the USB flash drive in this example, and used for distinguishing the two users sharing the one USB flash drive. When connected with the USB flash drive, the blood pressure measurement device (300) may ask each user to provide the assigned key to determine which main data folder the measurement data may be written to.

```
TABLE 1
Main folder:
[Health_care_Info_User_1]
    Personal_profile.txt
    Alarm_settings.txt
    [BP subfolder]
        Blood pressure data
        Device_config.txt ← configuration for individual user
    [Other subfolders]
        Data
[Health_care_Info_User_2]
    Personal_profile.txt
    Alarm_settings.txt
    [BP subfolder]
        Blood pressure data
        Device_config.txt ← configuration for individual user
    [Other subfolders]
        Data
[Firmware folder]
    [BP subfolder]
        Firmware ← updated program ready to download to BP device
        Firmware configuration ← configuration for all users
    [Other subfolders]
        Data
[Software folder]
    Software ← run on computer to managing and analyzing blood
pressure data on removable memory
    Software configuration
========================
[other document folders]
    Other document files
```

The Personal_profile.txt file in Table 2 is an example for storing personal information about a user. The personal information may include data such as name, gender, height and weight of the user, and ID number, etc., and present in a format shown in Table 2 as an example. Content and format of the personal information may vary.

TABLE 2

| | |
|---|---|
| Name: | Family-name First_name |
| Gender: | Male or Female |
| Birthday: | yyyy-mm-dd |
| Height: | cm |
| Weight: | Kg |
| ID: | ID###### |
| Updated_on: | yyyy-mm-dd |

The Alarm_settings.txt file in Table 1 contains alarm setting information, which may have a format as shown in Table 3.

TABLE 3

| | |
|---|---|
| 08:00 | Measure_BP |
| 09:00 | Take_Medicine Crestor 5 mg |
| 15:00 | Measure_BP |
| 19:30 | Fitness_Exercise |
| 22:00 | Measure_BP |

The [BP Subfolder] under the main folders [Health_Care_Info_User_{—}1] and [Health_Care_Info_User_{—}2], respectively, in Table 1, is created for storing blood pressure measurement, and other related data, such as measurement time, systolic pressure, diastolic pressure, heart rate, body temperature, Saturation of Oxygen (SaO₂), room temperature and humidity, and other measurement states, etc.

Configuration information for the blood pressure measurement device (300) may also be stored in the removable memory device (328). One type of configuration information may be information that individualizes specific configurations of the blood pressure measurement device (300) with respect to a particular user, such as the Device_config.txt under the main folders [Health_Care_Info_User_{—}1] and [Health_Care_Info_User_{—}2], respectively, in Table 1. Each user, i.e., User 1 and User 2, has a separated configuration file. The individualized configuration may allow each user to have personalized settings on a blood pressure measurement device he/she uses. The individualized configurations may define whether certain measurement is to be automatically performed or stored, such as the room temperature, or whether the heart beat sound is beeping during BP measurement. When connected with the removable memory device (328), firmware running on the blood pressure measurement device (300) may allow the blood pressure measurement device (300) to automatically read in the configuration information, and perform the configuration.

The removable memory device (328) may also store the latest firmware in the [Firmware folder] in Table 1, for upgrading programs on the blood pressure measurement device (300). Thus firmware on the blood pressure measurement device (300) may get upgraded when connected with the removable memory device (328). Under the [Firmware folder] in Table 1 may also be stored firmware configuration for configure generally the blood pressure measurement device (300), e.g., definitions of the format and file structure for saving the blood pressure measurement, and/or other related data.

When multiple users use the blood pressure measurement device (300), each may also have his/her own removable memory device, such that each may connect his/her removable memory device with the blood pressure measurement device (300) for measuring blood pressure and building up his/her own blood pressure record. In this case, there may be only one main folder for archiving various health related data on the removable memory device of each single user. The blood pressure measurement device (300) may identify each user using the personal information stored on each removable memory device, and directly write blood pressure measurement, measurement states and other related data into the blood pressure subfolder on each removable memory device.

Software programs may be stored in the removable memory device (328), e.g., the [Software folder] in Table 1, for manipulating the blood pressure and other related data stored in the blood pressure measurement device (300) or the removable memory device (328). The manipulating software programs may run from the removable memory device (401) that is connected with a computer, or from a computer hard-drive which is either connected with the removable memory device (328) or the blood pressure measurement device (300). The software programs may obtain data stored in the removable memory device (328) or the blood pressure measurement device (300), and allow a user to view, edit, add, backup or output, etc. the blood pressure and other related data, and to perform analysis regarding the user's blood pressure conditions. The analysis may correlate the blood pressure measurements, measurement states, and other related data, and give a general comparison, suggestions or alerts. The user may also view analysis results on the computer, save the analysis results into the removable memory device (328) or the computer.

Each of the embodiments described in FIGS. 1, 2 and 3, provides different functions and features for a blood pressure measurement device, e.g., providing measurement state, displaying multiple sets of blood pressure measurements, or providing interface for the blood pressure measurement device to communicate with other external devices. However, a blood pressure measurement device may have any or any combination of the features. For example, a blood pressure measurement device may provide simultaneous display of two different sets of blood pressure measurements, and allow a subject to input measurement states for his/her blood pressure measurement, in addition to measuring blood pressure of the subject.

With the features described in FIGS. 1, 2 and 3, a variety of data may be provided by a blood pressure measurement device. The data may include, but not limited to, blood pressure measure data, measurement states, and other related data.

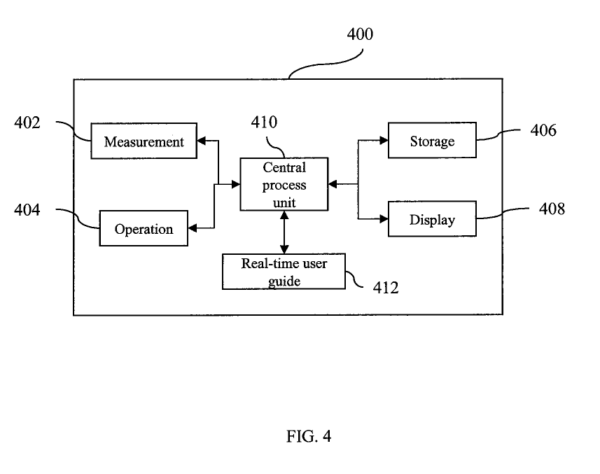

A blood pressure measurement device with one or more features described above may further provide real-time user guidance guiding a subject to use the blood pressure measurement device, as illustrated in FIG. 4. FIG. 4 illustrates an embodiment of a blood pressure measurement device (400). Similar to the blood pressure measurement device in the embodiment of FIG. 1, the blood pressure measurement device (400) may comprise a measurement component (402), an operation component (404), a storage component (406), a display component (408) for displaying simultaneously two sets of blood pressure measurement data, a CPU (410), and a real-time user guide component (412).

The real-time user guide component (412) may provide real-time guidance helping a user use and/or operate the blood pressure measurement device (400). The real-time guidance may be in video format including texts, graphics, etc or audio format including voice, music, etc., and may be presented by a display screen or a speaker of the blood pressure measurement device (400), if applicable. For example, when a user finishes one measurement of blood pressure, the real-time user guide component (412) may display messages on a display screen such as “press button 1 if you want to save the blood pressure measurement, or press button 2 if you want to have another measurement”, and/or a similar voice instruction may be given to the user. Thus a user may simply follow the guiding instructions and accomplish operations he/her needs.

The real-time guidance may also include alarms set up by a user. An alarm may be used to remind the user for certain activities, such as measuring blood pressure, doing exercises, or taking medicine. The alarms may help the user establish regular blood pressure records and keep monitoring or improving health conditions. Alarms may be set up by operating operational keys of the blood pressure measurement device (400), or an external device that may be removably connected with the blood pressure measurement device (400) via a bi-direction multifunctional port applicable, and alarm settings may be saved in the storage device (408), or a removably memory device if connected with the blood pressure measurement device (400).

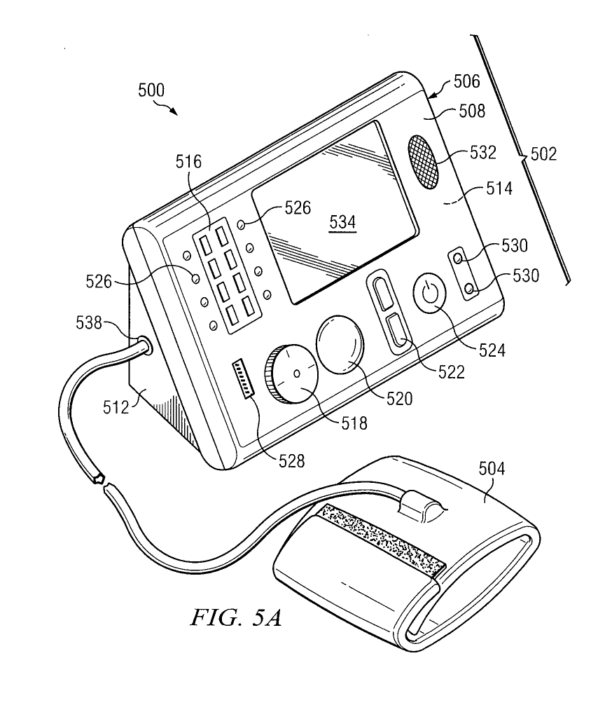

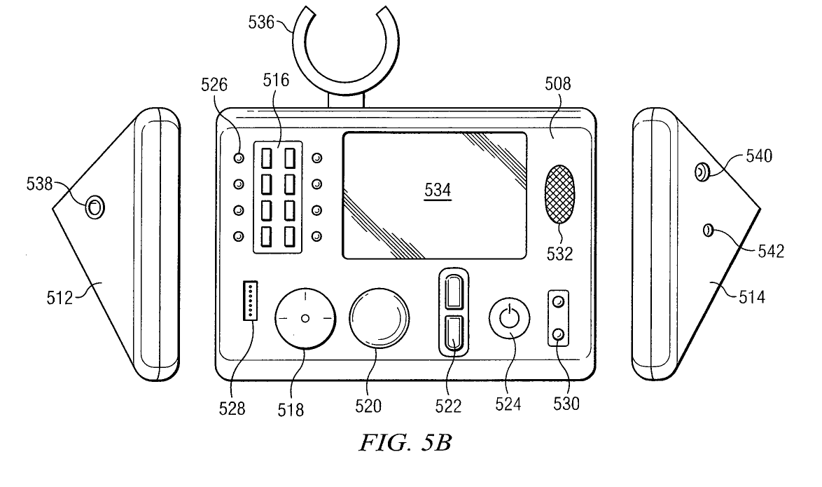

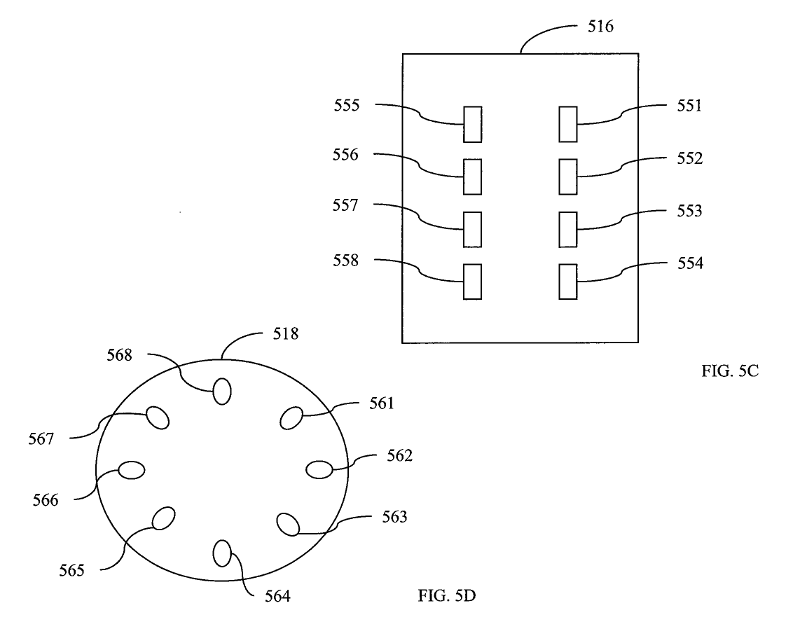

Referring to FIGS. 5A, 5B, 5C and 5D, an embodiment of a blood pressure measurement device (500) providing some of the features described above is illustrated according to the present invention. As illustrated in FIG. 5A, the blood pressure measurement device (500) includes a body unit (102), providing, controlling and managing various operations and functions of the blood pressure measurement device (500); a cuff (504) removably or integrally connected with the main body (502), applying pressure for measuring blood pressure of a subject. The main body (502) may include a housing (506), and a plurality of internal components inside the housing (506), which providing functionalities of the blood pressure measurement device (500).

The housing (506) may include a front panel (508), a left side panel (512) and a right side panel (514). The front panel (510) may include a plurality of input keys (516), (518), (520), (522) and (524) for operating the blood pressure measurement device (500); a plurality of LEDs (526) beside the keys (516); a bi-direction multifunctional port (528) that may be used to connect various external devices, such as a removable memory device, a keyboard, a printer, or a computer, extending functionalities of the blood pressure measurement device (500), and the bi-direction multifunctional port (528) may also be connected to a hub, which may allow multiple external devices to be connected with the blood pressure measurement device (500) at the same time; two LED indicators (530) indicating different on-going events, such as reminding a user to take medicine or measuring blood pressure; a speaker (532); a display screen (534) for displaying blood pressure measurement, measurement states, and other related data; and a cuff holder (536) for holding the cuff (504). The left side panel (512) has a cuff inlet (538) as shown in FIG. 5B, where the cuff is removably connected with the body unit (502). The right side panel (514) has a power socket (540), and an AUX port (542) for connecting applicable peripherals, such as a microphone, a temperature, humidity or SaO2 sensor.

The plurality of input keys (516), (518), (520), (522) and (524) may include a button section (516) that comprises multiple buttons, each button having a correspondent LED (526) indicating button selection; a mode key (518) for selecting different modes for operating the blood pressure measurement device (500); an Enter/Stop key (520) for confirming selection, starting or stop a measurement; a navigating key (522); and a power on/off key (524).

Each button in button section (516) may be configured to correspond to one function for operating the blood pressure measurement device (500). The buttons in section (516) may also be configured to select, or deselect various measurement states.

As illustrated by one embodiment in FIG. 5C, eight buttons are provided corresponding to eight measurement states shown in Table 4. A user may select one or more measurement states by operating the buttons, any combination of the buttons, or may not select any measurement state. If a user just took medication before he/she starts to measurement blood pressure, he/she may press button 555 to record this measurement state. The mode key (518) may be used to select the level or severity of each measurement state, such as extremely/very/kind-of hungry, drink a-lot-of/some/a little coffee, etc. When a button in section (516) is pressed, an LED (526) beside the button may be turned on indicating that a function or a measurement state represented by this button is selected.

TABLE 4

| Button | Function |
|---|---|
| 551 | Hungry |
| 552 | Bad mode |
| 553 | Sleepy |
| 554 | Dizzy |
| 555 | Take medication |
| 556 | Take caffeine |
| 557 | Take alcohol |
| 558 | After exercise |

The mode key (518) may be a rotary switch, a slide bar, or an applicable component that supports multiple positions, and each function may be selected by rotating the rotary switch or sliding the bar to a certain position, such as position (561) in FIG. 5D. FIG. 5D is an example of a rotary switch (518) with various functions supported. Functions corresponding to each position on the rotary switch are shown in Table 5.

TABLE 5

| Position | Function |
|---|---|
| 561 | Select a target user |
| 562 | Measure blood pressure |
| 563 | Review records |
| 564 | Review statistical data of blood pressure |
| 565 | Edit data |
| 566 | View or edit user information |
| 567 | View or edit alarm information |
| 568 | View or edit operational settings of a blood |
| | pressure measurement device |

Various data that may be provided by the blood pressure measurement device (500) may include blood pressure measurement, measurement states, and other related data. The blood pressure measurement may include systolic pressure, diastolic pressure, and heart rate. The other data that may be recorded in the blood pressure measurement device (500) may included a user's personal information, alarms, notes, and information that is related to the measurement of the blood pressure. The data may be selected using the input keys or measured by the blood pressure measurement device (500), or input into the blood pressure measurement device (500) via an external device.

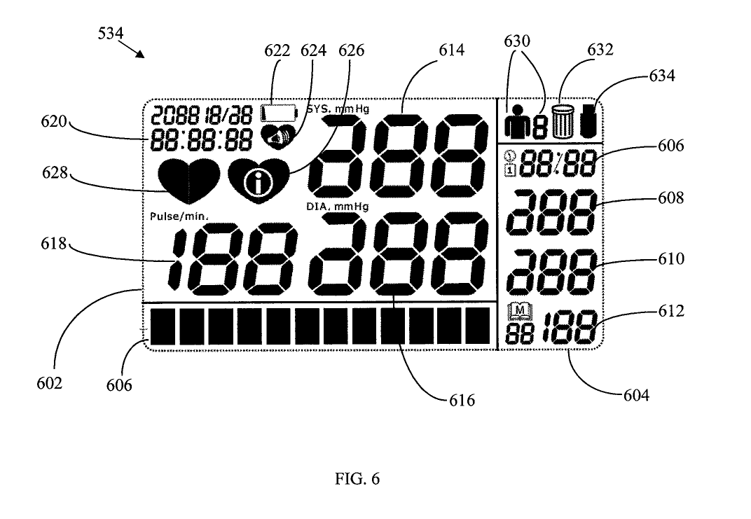

FIG. 6 illustrates one embodiment of display of the blood pressure measurement device (500) on the screen (534). A display may be provided for each user of the blood pressure measurement device (500). The screen (534) may comprise display sections (602), (604), and (606). Sections (602) and (604) display the current and previous blood pressure measurements, respectively, at the same time. This feature may be called a dual measurement display, which makes it convenient and simple for a user to compare the current and previous blood pressure measurements.

The previous blood pressure measurement includes previous measurement time (606) (month/day, or hour:Minute), a systolic pressure value (608), a diastolic pressure value (610), and a heart rate value (612). The current blood pressure measurement includes a systolic pressure value (614), a diastolic pressure value (616), and a heart rate value (618). As shown in FIG. 6, the display may also include a date and time display (620), displaying current date and time that may be updated every one second; a battery power indicator (622); a heartbeat sound on/off indicator (624); a blood pressure measurement error or warning indicator (626), that will turn on when an error is detected during measurement, or when hypertension or arrhythmia is detected; a heartbeat indicator (628); a target user indicator (630); a Trash-Can indicator (632); and a removable external device connection indicator (634).

The section (606) may be an on-screen message bar used to display messages, e.g., showing instruction messages to guide users to operate the blood pressure measurement device (100), or interact with users. In one example, a message bar (606) may be a dot-matrix display. The message bar (606) may display current mode selections and possible next actions. For example, the message bar (606) may display a message like “Start to measure the blood pressure” when a mode of “Measure blood pressure” is selected; or a message “Use the up/down arrows to select a target user” when a mode of “Select a target user” is selected.

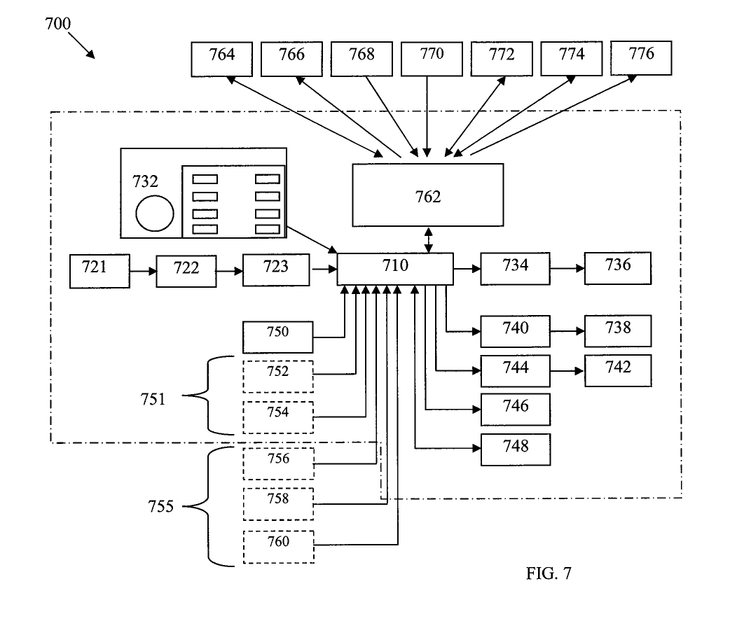

FIG. 7 is a block diagram (700) illustrating components of the blood pressure measurement device (500) according to one embodiment of the present invention. As illustrated in FIG. 7, a Central Processing Unit (CPU) (710) centrally controls and monitors each components of the blood pressure measurement device (500). A pressure sensor (721) detects pressure variations in the cuff (504), and outputs the cuff pressure signal to a filter (722). The filtered signal is then converted to a digital signal via an analog-to-digital converter (723), and sent to the CPU (710) for processing. A component (732) for controlling operational keys on the front panel (508) in FIG. 5 may translate a key selection into a signal and send to the CPU (710) for taking correspondent actions.

The CPU controls a display driver (734) to display data via a display screen (736), such as an LCD screen. A pump (738) and a valve (742) are driven by a pump driver (740) and a valve driver (744), respectively, controlling pumping and releasing of air in the cuff (504). The CPU (710) also controls a plurality of LEDs (746), such as the LEDs (526 and 530) in FIG. 5, to act accordingly based on settings or responses of the blood pressure measurement device (500). The blood pressure measurement, measurement states, and all other related data may be stored in an embedded memory (748) and/or a removable memory if applicable, and displayed on the display screen (736).

A real-time clock component (750) may be provided inside the blood pressure measurement device (500) to track time. The time, e.g., time in (620) of FIG. 6, may be displayed on the display screen (736) and is updated every one second via control of the CPU (710). The blood pressure measurement device (500) may further include other internal components for providing various measurements. For example, a humidity sensor (752) may be provided in the blood pressure measurement device (500), which measures humidity of the environment; and a room temperature sensor (754) may be provided to measure the current room temperature. The humidity or temperature measurements may then be displayed, manually or automatically, in the display screen (736), or stored in the embedded memory (748) or in the removable memory device.

In addition, the blood pressure measurement device (500) may be extended to include some exterior components or devices for measurement or other purposes. A body temperature sensor (756), a SaO2 sensor (758), or a room temperature sensor (760) may be connected with the blood pressure measurement device (500) via the AUX port (542), and operation of these exterior components may be controlled by the CPU (710). The internal components (751) or the exterior components (755) are optional components.

The blood pressure measurement device (500) may further include an interfacing component (762), which may comprise a bi-direction multifunctional port (528) in FIG. 5A. The interfacing component (762) may provide communication between the blood pressure measurement device (500) and a removable memory device (764), a printer (766), a keyboard (768), a microphone (770), a computer (772), another blood pressure measurement device (774), or another display screen (776).

Software programs may be provided on the blood pressure measurement device (500) to enable various functions in addition to measurement of blood pressure. The software programs provided and running on the blood pressure measurement device (500) may be referred to as “firmware” for description purpose in the following, and use of the term “firmware” is not to be construed as limiting. Functions of the firmware may allow to set or select a target user before blood pressure measurement. That is, multiple users may use the blood pressure measurement device (500), and may measure and store the measurements, measurement states, and other related data in the embedded memory (748) or in the removable memory device (764) corresponding to respective users.

The firmware may also allow a user of the blood pressure measurement device (500) to enter and store his/her personal information, alarms, or other information. The personal information may include data such as name, gender, height and weight of the user, etc. The alarms may be input to remind a user for certain activities, such as measuring blood pressure, or taking medicine.

The information may be entered to the blood pressure measurement device (500) through the keys on the front panel (508), e.g., rotary switch (518) may be set up to enter letters or numbers; or be entered using external devices that may be connected via the bi-direction multifunctional port (528), such as audio information is entered via the microphone (770), or the computer (772), or text information entered via the keyboard (768) or the computer (772). The information may further be directly written into the blood pressure measurement device (500) by the computer (772) or by other applicable means. The information may be displayed on the message bar (606), the whole screen (534), or by making sound, beeping, or flashing according to type of the information.

Functions of the firmware may allow the blood pressure measurement device (500) to display two sets of blood pressure measurement on the display screen (534), such as the current and latest blood pressure measurement. When blood pressure measurement device (500) is powered up, the section (604) may display the latest measurement of a last selected user. If a removable memory device, e.g., a USB flash drive is connected with the blood pressure measurement device (500), the section (604) may display the last measurement stored on the USB flash drive. When a user measures the blood pressure, the section (602) may display progress of measurement, and then display the current measured result. If the user measures blood pressure one more time, the “currently measured” result will be moved from section (602) to the section (604), and the section (602) may display the progress of a new measurement of blood pressure.

Functions of the firmware may further allow users to set up operational settings of the blood pressure measurement device (500), including, but not limited to, record data manually or automatically, beep or not beep when detecting heartbeat, and set up date or time; to review data recorded in the blood pressure measurement device (500), such as blood pressure measurements, measurement states, personal information, or alarms, etc; to review statistical data, such as average systolic and diastolic pressure during a particular period of time; to delete data stored in the embedded memory (748) or in the removable memory device (764); or to select to measure some measurement states, such as room temperature or humidity by a temperature sensor or a humidity sensor, respectively, that may be connected with the blood pressure measurement device (500) or embedded in the blood pressure measurement device (500).

Moreover, functions of the firmware may provide real-time guidance helping users use and/or operate the blood pressure measurement device (500). The blood pressure measurement device (500) may use the message bar (606) to display the guidance, or the speaker (532) to give audible voice instruction. For example, when a user wants to select a target user, and rotates the rotary switch (518) to the corresponding position (561), the message bar may display instructions like “Use up/down arrow to select a target user, and press the Enter/stop key for selection”, and/or a similar voice instruction may be given to the user. Thus a user may simply follow guiding instructions and accomplish operations he/her needs.

Alarms may also be set up by operating input keys of the blood pressure measurement device (500), or an external device that may be removably connected with the blood pressure measurement device (500) via the bi-direction multifunctional port (528), and alarm settings may be saved in the embedded memory (748), or the removably memory device (764) if connected with the blood pressure measurement device (500).

The blood pressure measurement device (500) may continuously compare the current time, e.g., time read from real-time clock component (750), to the alarm settings. When the current time matches any alarm time in the alarm settings, the blood pressure measurement device (500) device may generate visible and/or audible alarms, such as generating beeping, or flashing LED.

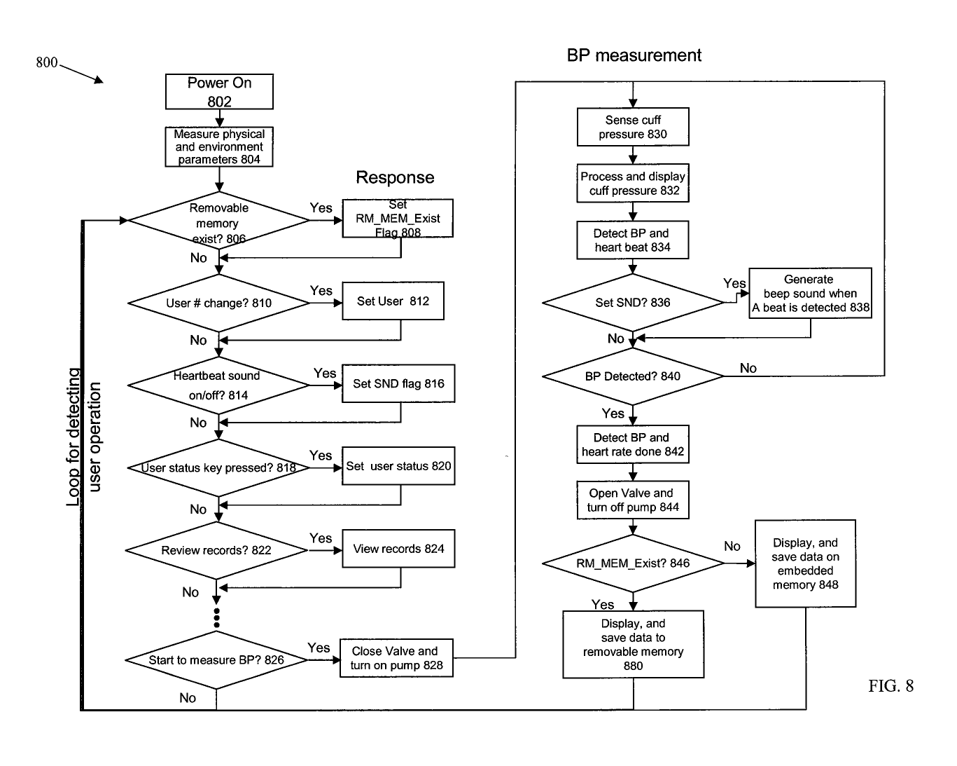

FIG. 8 is a flow chart (800) illustrating one embodiment of how the blood pressure measurement device (500) works according to the present invention. When power is turned on in (802), the blood pressure measurement device (500) may automatically measure some physical or environmental parameters in (804), such as body temperature and/or SaO2, room temperature and/or humidity, if devices for measuring such parameters are available. The blood pressure measurement device (500) may repeatedly check its working modes, as shown by the determination blocks (806), (810), (814), etc., and take actions accordingly. The steps of various mode checking and correspondent actions taken may not be performed in a specific order, but may depend on users' operations. There may be many other steps that are not illustrated in FIG. 8.

Before measuring the blood pressure, the blood pressure measurement device (500) may determine whether a removable memory device is connected in (806), and set up a flag in (808) if there is a removable memory device connected; determine whether a target user is changed in (810) and set up the target user if needed in (812); determine whether heartbeat sound is to be turned on or off in (814), and set up a flag in (816) if the sound is turned on; determine whether measurement states are selected or selected to be measured in (818), and set or measure the measurement states in (820) if the determination is yes; determine whether a user wants to review records stored in (822), and enters record view process in (824) if the user chooses to review the records in the blood pressure measurement device (500).

When the blood pressure measurement device (500) detects in (826) that it is in a mode of measuring blood pressure, it may close the valve and begins pumping air into the cuff by turning on the pump in (828). The blood pressure measurement device (800) may then sense cuff pressure in (830), process and display the cuff pressure in (832), and detect blood pressure and heart rate of the user in (834). If the heartbeat sound is turned on in (836), then a beep sound may be generated each time when a heart beat is detected in (838).

If no blood pressure is detected in (840), the blood pressure measurement device (500) may go back to step (830) and repeat the measurement process. If blood pressure is detected in (840), the blood pressure measurement device (500) ends the blood pressure measurement in (842), opens the valve and turns off the pump in (844).

If there is not a removable memory device connected with the blood pressure measurement device (500) in (846), the blood pressure measurement device (500) may display measurement results on the display screen, and save blood pressure measurement, measurement states and other related data in the embedded memory for the selected target user in (848). If there is a removable memory device connected with the blood pressure measurement device (500) in (846), the blood pressure measurement device (500) may display measurement results on the display screen, and save blood pressure measurement, measurement states and other related data in the removable memory device in (850).

If the blood pressure measurement device (500) is not in measuring blood pressure mode or finishes the blood pressure measurement, it may go back to step (806), and wait for users' operations to take correspondent actions. The blood pressure measurement device (500) may be turned off automatically if it does not detect users' operation for a predetermined time period, such as 2 minutes.

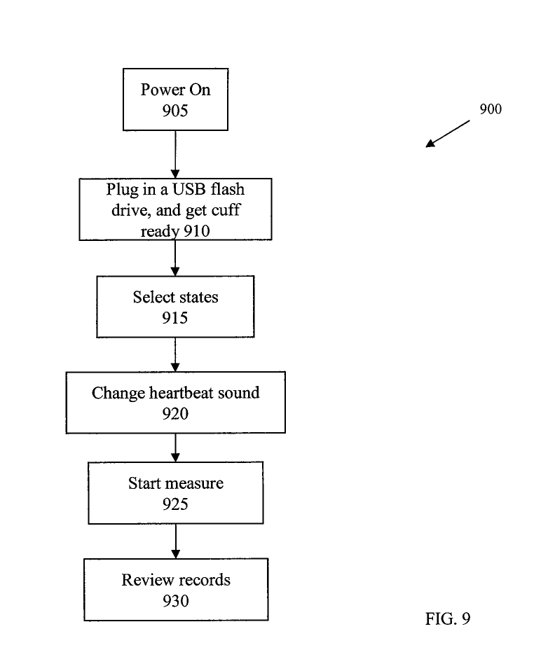

FIG. 9 is a block diagram (900) illustrating one embodiment of using the blood pressure measurement device (500). Referring also to FIGS. 5 and 6, with the buttons in section (516) and the rotary switch (518) set up as in FIGS. 5C, 5D and Table 4 and 5, a user may first turn on the blood pressure measurement device (500) by pressing the power on/off key (524) in step (905), wrap the cuff (504) in a right position, and plug in a USB flash drive in the bi-direction multifunctional port (528) in step (910). The blood pressure measurement device (500) detects the USB flash drive, and may display a message on the message bar (606) “USB drive connected”. The blood pressure measurement device (500) may also identify the target user by reading personal information stored in the USB flash drive.

The user may select measurement states in step (915) by pressing the buttons in the section (516). For example, if it is in the morning, and the user just performed some exercises and hasn't had breakfast yet, the user may press button (551) and (558). The blood pressure measurement device (500) records the measurement states. If a body temperature sensor is connected to the blood pressure measurement device (500) via the AUX port (542), the body temperature data will be measured and stored together with the blood pressure measurement data.

The user may also want to turn the heartbeat sound on. In step (920), the user may rotate the rotary switch (518) to the position of (568) for setting the blood pressure measurement device (900), find the sound setting by clicking the navigating key (522), and turn on the sound, by clicking the Enter/Stop key (520).

The user may then start measure blood pressure by rotating the rotary switch (518) to position (562) in step (925). The screen (534) may show the inflating cuff pressure, indicators (530) may flash and the blood pressure measurement device (500) may beep for every heart pulse detected. The message bar (606) may show a message like “measuring . . . ”. The measurement result may be shown on the section (602) of screen (534), and previous blood pressure measurement may be shown on the section (604). The user may continue or stop the measurement by clicking the Enter/Stop key (520). The blood pressure measurement, measurement states, and other related data may be stored in USB flash drive.

In step (930), the user may view the stored records by rotating the rotary switch (518) to the position (563). The message bar (606) may show a message like “view records”. The user may view all data by use of the navigating key (522).

Functions corresponding to each button in the section (516) or the key (518) may be customized or reconfigured to adapt to various user needs or conditions. Buttons in the section (516) and the key (518) may further be combined to provide more functional options for the blood pressure measurement device (500).

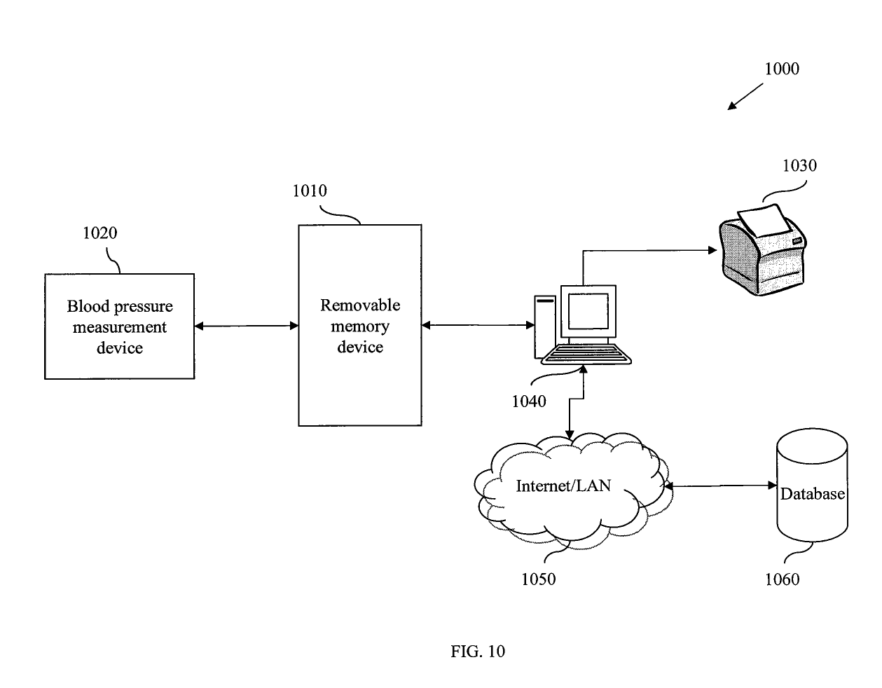

Inclusion of one or more features described above, such as connecting with removable external devices via a bi-direction multifunctional port, greatly extends functions provided by a blood pressure measurement device. With such a blood pressure measurement device, a comprehensive system may be formed for integrating and managing blood pressure and related data. FIG. 10 is a schematic diagram illustrating a system (1000) structure for integrating and managing blood pressure and related data according one embodiment of the present invention.

The system (1000) comprises a removable memory device (1010), which is similar to the removable device (328) in FIG. 3; a blood pressure measurement device (1020), e.g., the blood pressure measurement device in FIG. 3; a printer (1030) and a computer (1040). The blood pressure measurement device (1020) provides blood pressure and other related data, and may write to or read from the removable memory device (1010) when connected with the removable memory device (1010). The removable memory device (1010) integrates the blood pressure and other related data provided by the blood pressure measurement device (1020).

The removable memory device (1010) may be connected with the computer (1040), and data stored in the removable memory device (1010) may be manipulated through the computer (1040). Manipulation of the data may include, but not limit to, add, delete, edit, display, backup, output, analyze the data, and read from or write to the removable memory device (1010). The computer (1040) may be connected to an Internet or a Local Area Network (LAN) (1050). Data stored in the removable memory device (1010) may be stored remotely in a database server (1060) on the Internet or LAN (1050), and printed out via the printer (1030), locally or remotely. Firmware may be provided to run from the blood pressure measurement device (1020), or software programs may be provided to run from the computer (1040) or the removable memory device (1010), respectively, implementing functions described above for the system (1000).

In the system (1000), the removable memory device (1010) may also be connected with other health related external devices, such as a fitness training apparatus (e.g., a treadmill), body weight, or body fat analyzer, storing and exchanging health related data. Firmware may be provided on the other health related external devices to allow them to provide the health related data, write or read the health related data to or from the removable memory device (1010) in a particular format and structure that is accessible by other health related external devices, including the blood pressure measurement device (1020) or the computer (1040). Thus the removable memory device (1010) may store and integrate various health related data, which may provide a complete picture of health conditions of a user. Furthermore, exchange of the health related data, between health related external devices, or between a health related external device and the computer (1040), may greatly improve the efficiency for using the obtained health related data. The system (1000) may benefit many people in a variety of fields, such as doctors, nutritionists, fitness trainers, health care advisors, lifestyle advisors, healthcare device end users, and fitness equipment users, etc.

The previous description of the disclosed embodiments is provided to enable those skilled in the art to make or use the present invention. Various modifications to these embodiments will be readily apparent to those skilled in the art and generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A blood pressure measurement device, comprising:
a measurement component for measuring blood pressure; and
a display component for displaying the blood pressure data measured;
wherein the display component is adapted to display at least two sets of blood pressure measurement data measured at different date or time simultaneously.

2. The blood pressure measurement device in claim 1, wherein the at least two sets of blood pressure measurement comprise current blood pressure measurement and the latest blood pressure measurement.

3. The blood pressure measurement device in claim 1, wherein the blood pressure measurement comprises systolic blood pressure, diastolic blood pressure, heart rate value, and measurement time.

4. The blood pressure measurement device in claim 1, further comprising a real-time user guide component, adapted to provide real-time guidance for users to use or operate the blood pressure measurement device.

5. The blood pressure measurement device in claim 4, wherein the real-time user guide component provides video or audio guidance or combination of video and audio guidance.

6. A blood pressure measurement device, comprising:
a measurement component for measuring blood pressure;
a measurement state control component for recording and storing at least one measurement state related to the measurement of the blood pressure; and
means for providing the at least one measurement state.

7. The blood pressure measurement device in claim 6, wherein the at least one measurement state is provided by at least one operational key.

8. The blood pressure measurement device in claim 6, wherein the at least one measurement state is provided by a plurality of operational keys, the plurality of operational keys adapted to select level or severity of the at least one measurement state.

9. The blood pressure measurement device in claim 6, wherein the at least one measurement state is provided by at least one external device removably or integrally connected with the blood pressure measurement device.

10. The blood pressure measurement device in claim 9, further comprising means for connecting the at least one external device removably with the blood pressure measurement device.

11. The blood pressure measurement device in claim 10, wherein the connection port comprises a Universal Series Bus (USB) port.

12. The blood pressure measurement device in claim 9, wherein the at least one external device comprises a device selected from the group consisting of a computer, a keyboard, a microphone, and a measurement device.

13. The blood pressure measurement device in claim 9, wherein measurement device comprises a device selected from the group consisting of a room temperature reader, a body temperature reader, and a room humidity reader.

14. The blood pressure measurement device in claim 6, further comprising a real-time user guide component, adapted to provide real-time guidance for users to use or operate the blood pressure measurement device.

15. The blood pressure measurement device in claim 14, wherein the real-time user guide component provides video or audio guidance or combination of video and audio guidance.

16. A blood pressure measurement device, comprising:
a measurement component for measuring blood pressure;
an interfacing component, comprising a bi-direction multifunctional port for exchanging blood pressure data measured between the blood pressure measurement device and at least one external device.

17. The blood pressure measurement device in claim 16, wherein the at least one external device comprises a device selected from the group consisting of a computer, a removable memory device, a printer, a key board, a blood pressure measurement device, a microphone, and a display screen.

18. The blood pressure measurement device in claim 16, wherein data related to the blood pressure measurement is stored in a memory device that is removably connected with the blood pressure measurement device by way of the bi-direction multifunctional port, the data in the removable memory device stored in a specific format and file structure.

19. The blood pressure measurement device in claim 18, wherein the removable memory device comprises a non-volatile memory product.

20. The blood pressure measurement device in claim 18, wherein the removable memory comprises a flash drive or flash memory.

21. The blood pressure measurement device in claim 16, further comprising a real-time user guide component, adapted to provide real-time guidance for users to use or operate the blood pressure measurement device.

22. The blood pressure measurement device in claim 21, wherein the real-time user guide component provides video or audio guidance or combination of video and audio guidance.

23. A blood pressure measurement device, comprising:
a measurement component for measuring blood pressure;
a display component for displaying the blood pressure data measured;
a measurement state control component for recording and storing at least one measurement state related to the measurement of the blood pressure;
an interfacing component, comprising a bi-direction multifunctional port, for exchanging blood pressure data measured between the blood pressure measurement device and at least one external device; and
means for providing the at least one measurement state;
wherein the display component is adapted to display at least two sets of blood pressure measurement data measured at different date or time simultaneously.

24. The blood pressure measurement device in claim 23, wherein the at least two sets of blood pressure measurement comprise current blood pressure measurement and the latest blood pressure measurement.

25. The blood pressure measurement device in claim 23, further comprising a real-time user guide component, adapted to provide real-time guidance for users to use or operate the blood pressure measurement device.

26. The blood pressure measurement device in claim 23, wherein the at least one measurement state is provided by at least one operational key.

27. The blood pressure measurement device in claim 23, wherein the at least one measurement state is provided by a plurality of operational keys, the plurality of operational keys adapted to select level or severity of the at least one measurement state.

28. The blood pressure measurement device in claim 23, wherein the at least one measurement state is provided by at least one external device removably or integrally connected with the blood pressure measurement device.

29. The blood pressure measurement device in claim 28, wherein the at least one external device comprises a device selected from the group consisting of a computer, a keyboard, a microphone, and a measurement device.

30. The blood pressure measurement device in claim 23, wherein the at least one external device comprises a device selected from the group consisting of a computer, a removable memory device, a printer, a key board, a blood pressure measurement device, a microphone, and a display screen.

31. The blood pressure measurement device in claim 23, wherein data related to the blood pressure measurement is stored in a memory device that is removably connected with the blood pressure measurement device by way of the bi-direction multifunctional port, the data in the removable memory device stored in a specific format and file structure.

32. The blood pressure measurement device in claim 31, wherein the removable memory device comprises a non-volatile memory product.
